(19) United States
(12) Patent Application Publication
(10) Pub. No.: US 2018/0327430 A1
(43) Pub. Date: Nov. 15, 2018

(54) Method for Preparing Methoxyboranes and for Producing Methanol

(71) Applicant: Commissariat a l'Energie Atomique et aux Erergies Alternatives, Paris (FR)

(72) Inventors: Clément Chauvier, Paris (FR); Thibault Cantat, Issy les Moulineaux (FR)

(73) Assignee: Commissariat a l'Energie Atomique et aux Energies Alternatives, Paris (FR)

(21) Appl. No.: 15/774,851

(22) PCT Filed: Nov. 8, 2016
(86) PCT No.: PCT/EP2016/077010

§ 371 Date: May 9, 2018

(30) Foreign Application Priority Data
Nov. 10, 2015 (FR) 1560747

(51) Int. Cl. C07F 5/02; C07C 29/12; C07C 29/10; C07C 29/09

(52) U.S. Cl. CPC C07F 5/027; C07C 29/12; C07C 31/04; C07C 29/10; C07C 29/095; C07F 5/025

(57) ABSTRACT

The present disclosure relates to a method for preparing methoxyboranes by dismutation of formic acid or at least one of the derivatives thereof or a mixture of formic acid and at least one of the derivatives thereof, in the presence of an organoborane, and optionally an organic or inorganic base.

The present invention relates to a method for preparing methoxyboranes by dismutation of formic acid or at least one of the derivatives thereof or a mixture of formic acid and at least one of the derivatives thereof, in the presence of an organoborane, and optionally an organic or inorganic base.

The invention also relates to a method for producing methanol comprising a step for preparing a methoxyborane according to the method of the invention and a step for hydrolysis or protonolysis of the methoxyborane into methanol.

The invention further relates to the use of methoxyboranes obtained by the method of the invention for the production of methanol, as catalysts in allylation reactions, as reagents in Suzuki coupling reactions, and as a reagent for fine chemistry or for heavy chemistry.

The methanol produced by hydrolysis or protonolysis of the methoxyboranes formed by the method of the invention can be used as a fuel or additive in combustion engines, as a fuel in direct methanol fuel cells, as a means for storing hydrogen, or as a reagent for fine chemistry.

Methanol, CH₃OH (MeOH), is currently at the heart of an energy strategy in that it a leading candidate for replacing fossil fuels such as petrol, diesel or gas. Indeed, methanol has high octane ratings, and can as such be used directly as a fuel in current engines. Furthermore, it can be used in Direct Methanol Fuel Cells (or DMFCs), i.e. fuel cells where methanol is introduced as is as a fuel for generating electricity and thereby makes it possible to avoid the use of hydrogen gas H₂, characteristic of conventional fuel cells. A further major application sector for methanol is the production of chemical substances and particularly of olefins by means of the MTO (Methanol To Olefins) method or of acetic acid by carbonylation (Monsanto, and more recently, Cativa methods).

However, the applications mentioned above are essentially based on the use of methanol obtained from carbon-based fossil resources. More specifically, the main source of methanol at the present time is fossil methane (CH₄), which, by reforming, produces synthetic gas (CO+H₂) and the latter is finally converted into methanol. These reforming methods emit CO₂, a gas whose role in global warming is widely documented. Consequently, there is a growing need for cleaner methods for producing methanol, and particularly for methods enabling the reintegration of CO₂ into the energy cycle.

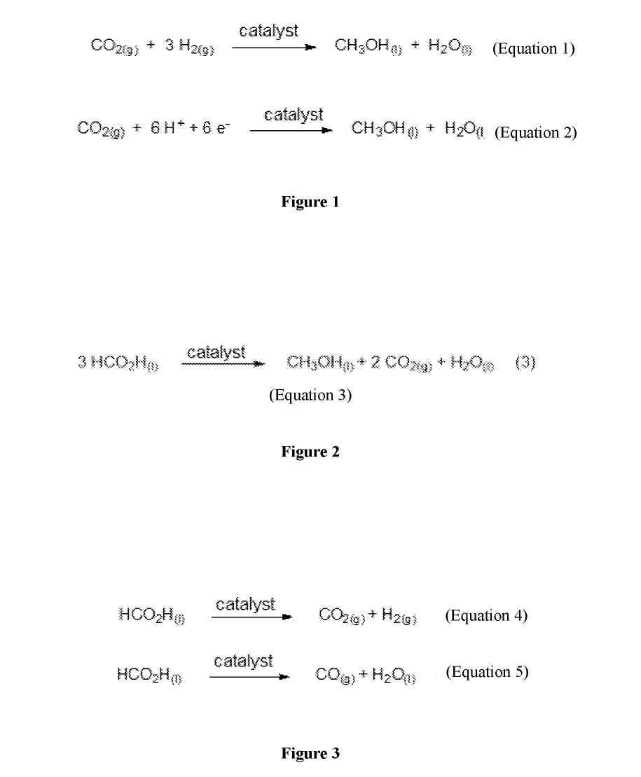

At the present time, two main approaches are explored to meet this requirement:
Hexaelectronic (with 6 electrons i.e. 3 hydrides) reduction of CO₂ into methanol by catalytic hydrogenation (FIG. 1—Equation (1)),
Hexaelectronic electro-reduction of CO₂ into methanol (FIG. 1—Equation (2)).

These two approaches involve a number of drawbacks, in particular: (i) a low selectivity, that is to say that a mixture of substances only containing a single carbon is obtained (i.e. formic acid, carbon monoxide, formaldehyde, etc.) in addition to methanol; (ii) low faradaic (electron transfer) yields due to the large number of electrons to be transferred effectively in a hexaelectronic reduction; (iii) the use of transition metals as catalysts, and particularly of noble metals such as ruthenium and iridium the price, toxicity and/or abundance thereof generally pose a problem.

1. Hydrogenation of CO₂ into Methanol

The hydrogenation reaction of CO₂ into methanol (equation (1) above) requires the use of catalysts suitable for facilitating the reaction. Indeed, although the thermodynamics are favourable (exergonic reaction, ΔG⁰=−40.9 kJ·mol⁻¹<0), the successive energy barriers leading to the formation of 3 C—H bonds are consequential and cannot be passed under acceptable temperature and pressure conditions.

The first catalyst capable of carrying out the reduction of CO₂ into methanol was reported by Sasaki et al. in 1993 [K.-i Tominaga, Y, Sasaki, M. Kawai, T. Watanabe, M. Saito, J. Chem. Soc, Chem. Commun. 1993, 629]. The latter used the complex Ru₃(CO)₁₂ in the presence of a halide salt KI to obtain methanol as a majority product in a mixture with other reduction products such as carbon monoxide CO or methane. The reaction is conducted in NMP (N-methyl-2-pyrrolidone) at 240° C. for 3 hours at 80 atmospheres (81.06 bar) of a CO₂:H₂ (1:3) mixture.

This reaction was updated in 2011 by the Sanford group in the USA [C. A. Huff, M. S. Sanford, J. Am. Chem. Soc. 2011, 133, 18122-18125]. The latter developed a complex catalytic system consisting of 3 different catalysts. Each of the three catalysts is capable of carrying out a different reduction step. The hydrogenation of CO₂ into formic acid HCOOH is catalysed by the ruthenium complex (PMe₃)₄Ru(Cl)(OAc). The formic acid produced in this step is then esterified in the presence of deuterated methanol used as a solvent and of a catalytic quantity of scandium triflate Sc(OTf)₃. Finally, the third complex (PNN)Ru(CO)(H) wherein PNN denotes 6□(di□tert□butylphosphinomethylene)□2□ (N,N□diethylaminomethyl)□1,6□dihydropyridine, enables the hydrogenation of methyl formiate into methanol. To ensure the compatibility between the three catalysts and the different species in presence in the reaction medium, the reaction is conducted in a double-chamber system at 135° C. for 16 hours to obtain methanol with TONs of 2.5 maximum. Pressures in CO₂ and H₂ of 10 and 30 bar respectively must be maintained to obtain satisfactory methanol yields.

To avoid the use to 3 metal catalysts to perform a single conversion, Leitner, Klankermayer et al. developed a catalytic system based on the use of the sole ruthenium complex (Triphos)Ru(TMM) (TMM=trimethylenemethane, Triphos=1,1,1-tris(diphenylphosphinomethyl)ethane) which, in the presence of an acid additive such as the acid triflimide (HNTf₂), makes it possible to obtain methanol with TONs ranging up to 221 [S. Wesselbaum, T. Vom Stein, J. Klankermayer, W. Leitner, Angew. Chem. Int. Ed. Engl. 2012, 51, 7499-7502]. The reaction is carried out in THF at 140° C. at a total pressure of 80 bar (60 bar H₂, 20 bar CO₂).

In the light of the difficulties encountered in the catalytic hydrogenation of CO₂ into methanol (low yield, harsh conditions, use of noble metals), a further strategy was envisaged to obtain methanol from CO₂, that is to say, functionalising CO₂ before carrying out the reduction steps. It is indeed known that the formation of carbamates or carbonates by reacting CO₂ with, respectively, an amine or an alcohol is an energetically favourable process. As such, numerous research groups are working on the hydrogenation of these derivatives of CO₂ rather than on the latter directly. Examples of hydrogenation reactions of carbonates or carbamates to obtain methanol are documented in recent reviews [E. Alberico, M. Nielsen, Chem. Comm. 2015, 51, 6714-6725; Y.-N. Li, R. Ma, L.-N. He, Z.-F. Diao, Catal. Sci. Technol. 2014, 4, 1498-1512.].

Moreover, it should be noted that besides the homogenous catalysts based on transition metals cited above, heterogenous catalysts such as Cu/ZnO are capable of carrying out the hydrogenation reaction of CO₂ into methanol [A. Goeppert, M. Czaun, J. P. Jones, G. K. Surya Prakash, G. A. Olah, Chem. Soc. Rev. 2014, 43, 7995-8048]. However, homogenous-phase catalysis is advantageous as it frequently means increased selectivity and milder operating conditions.

In parallel with the catalytic systems based on transition metals for the hydrogenation of CO₂, numerous research projects focus on the development of systems not containing metals and which are also capable of hydrogenating CO₂. The feasibility of such reactions without metals has been enabled by the emergence of frustrated Lewis pairs, i.e. compounds or mixtures containing a Lewis acid and a Lewis base which, due to the steric size thereof, cannot combine to form an adduct. It was, indeed, demonstrated in 2006 that the combination of an encumbered Lewis acid and Lewis base prevents the formation of a conventional Lewis adduct (hence the term “frustrated”) and consequently enables the fission of small molecules such as hydrogen onto a proton and a hydride [D. W. Stephan, Org. Biomol. Chem. 2008, 6, 1535-1539; G. C. Welch, R. R. San Juan, J. D. Masuda. D. W. Stephan, Science 2006.314, 1124-1126].

This concept was put to use in 2009 by O'Hare and Ashley who demonstrated for the first time that a system based on the combination of borane B(C₆F₅)₃ and the base tetramethylpiperidine (TMP) enables the reduction of CO₂ into methanol in the presence of hydrogen H₂. More specifically, the Lewis pair splits H₂ to produce the borohydride [TMPH]⁺[HB(C₆F₅)₃]⁻ which reacts with CO₂ to produce [TMPH]⁺[CH₃OB(C₆F₅)₃]⁻ which is a precursor of methanol before hydrolysis. This reaction is carried out in toluene at 160° C. for 6 days at CO₂ and H₂ pressures ranging from 1 to 2 atm to give maximum methanol yields of 25% [A. E. Ashley, A. L. Thompson, D. O'Hare, Angew. Chem. Int. Ed. Engl. 2009, 48, 9839-9843].

More recently, a similar approach was reported by Stephan, Fontaine et al. [M. A. Courtemanche, A. P. Pulis, E. Rochette, M. A. Legare, D. W. Stephan, F. G. Fontaine, Chem. Comm. 2015, 51, 9797-9800] using the intra-molecular frustrated Lewis pair 1-BR₂-2-NMe₂-C₆H₄ represented hereinafter in the presence of H₂ and CO₂. Nevertheless, the methanol yields are very low and a mixture of substances containing one carbon atom (i.e. formiates, acetals, etc.) is obtained.

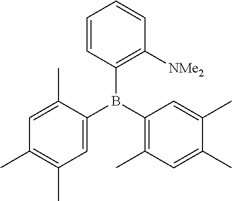

2. Hexaelectronic Electro- and Photo-Reductions of CO₂ into Methanol

Two major difficulties are encountered during the hexaelectronic electro-reduction of CO₂ into methanol, that is to say: a problem of selectivity for methanol, i.e. exclusively obtaining the latter and no other substances containing one carbon atom such as CO or formic acid. Furthermore, in the light of redox potentials, the selective reduction of CO₂ in the presence of protons is difficult as the latter may also be reduced into hydrogen at potentials similar to those of electro-reduction. As such, electrocatalysts must be developed both to overcome the high kinetic overpotentials associated with the transfer of 6 electrons, but also to ensure the selectivity of the reduction.

For this reason, there are few or no systems capable of carrying out 6e⁻ electro-reduction of CO₂ into methanol effectively with good selectivities. To this end, the research by Bocarsly et al. [G. Seshadri, C. Lin. A. B. Bocarsly, J. Electroanal. Chem. 1994, 372, 145-150] demonstrated as of 1994 that the pyridinium cation PyH⁺ enables the homogenous-phase catalysis of Pd electrode electro-reduction of CO₂ into methanol with faradaic yields of up to 30%. The benefit of this system being that the overpotentials applied are relatively low compared to numerous other systems. Further examples may be found in recent reviews such as those by Olah [A. Goeppert, M. Czaun, J. P. Jones, G. K. Surya Prakash, G. A. Olah, Chem. Soc. Rev. 2014, 43, 7995-8048] or Kenis [H.-R. M. Jhong, S. Ma, P. J. A. Kenis, Curr. Opin. Chem. Eng. 2013, 2, 191-199].

3. Hydrogenation and 2-Electron Electro-Reduction of CO₂ into Formic Acid
2-electron hydrogenation of CO₂ into HCOOH

This reaction is thermodynamically at a disadvantage (ΔG⁰=+32.9 kJ·mol⁻¹), and the addition of a base is required to shift the equilibrium towards the formation of the salt HCOO⁻, BaseH⁺.

The most active catalyst at the present time for this conversion is an iridium complex PNP-IrH₃ (PNP=2,6-bis(di-tert-butylphosphinomethyl)pyridine) reported by Nozaki et al. in 2009 [R. Tanaka, M. Yamashita, K. Nozaki, J. Am. Chem. Soc. 2009, 131, 14168-14169]. TONs of 3,500,000 and TOFs of 150,000 h⁻¹ were obtained in aqueous potash solution KOH at 120° C. after 48 hours of reaction at a total pressure of 60 bar (30/30 CO₂/H₂). The reaction product is then potassium formiate HCOOK, which, in the presence of a strong acid, may release formic acid HCOOH [C. Federsel, R. Jackstell. M. Beller, Angew. Chem. Int. Ed. Engl. 2010, 49, 6254-6257].

In 2012, Beller et al. [C. Ziebart, C. Federsel, P. Anbarasan, R. Jackstell, W. Baumann, A. Spannenberg, M. Beller, J. Am. Chem. Soc. 2012, 134, 20701-20704] demonstrated that the iron complex Fe(BF₄)₂.6H₂O placed in the presence of the ligand tris(2-(diphenylphosphino)phenyl)phosphine enables the hydrogenation of CO₂ into formic acid and into derived substances such as dimethylformamide (DMF) when dimethylamine is added to the reaction medium of methyl formiate when the methanol is solvent. TONs of approximately 2000 are obtained at 100° C. at total pressures of 60 bar (30/30 CO₂/H₂).

Dyson et al. [S. Moret, P. J. Dyson, G. Laurenczy, Nat. Commun. 2014, 5, 4017] describes a ruthenium-based catalyst which makes it possible to carry out the hydrogenation reaction of CO₂ in aqueous solution with no basic additive (mentioned above as being necessary to shift the equilibrium). At a total pressure of 100 bar (50/50 CO₂H₂), the complex RuCl₂(PTA)₄ (PTA=1,3,5-triaza-7-phosphaadamantane) carries out up to 159 TONs and formic acid HCOOH is obtained at a concentration of 0.1 M in water. If DMSO is used as a solvent, TONs of 475 may be reached.
2 e⁻ electro-reduction with of CO₂ into HCOOH

This strategy is currently the only economically viable electrochemical reduction process of CO₂. Indeed, formic acid and the corresponding formiates are currently the only substances containing one carbon atom suitable for being generated with a relatively low energy impact and with high faradaic yields. Moreover, the formic acid market is likely to grow dramatically due to the large number of applications of formic acid, particularly as a candidate for storing hydrogen for light applications, i.e. applications with a limited energy requirement such as mobile telephony [A. S. Agarwal, Y. Zhai, D. Hill, N. Sridhar, Chem Sus Chem 2011, 4, 1301-1310; X. Lu, D. Y. C. Leung, H. Wang, M. K. H. Leung, J. Xuan, Chem Electro Chem 2014, 1, 836-849]. It should be noted that current formic acid production is evaluated at 800 kT·year⁻¹.

Numerous systems based on homogenous or heterogenous catalysts are viable for 2-electron electro-reduction of CO₂ into formic acid, i.e. the faradaic yields obtained are greater than 80% with relatively large current densities. For example, when the working electrode is coated with a Pd layer, faradaic yields of 100% are obtained [B. I. Podlovchenko, E. A. Kolyadko, S. Lu, J. Electroanal. Chem. 1994, 373, 185-187]. Numerous examples along with a technico-economic analysis of the viability of the CO₂ to HCOOH process have been published.

4. Dismutation of Formic Acid into Methanol and CO₂

On the basis of all of the above, it appears that the formation of methanol directly from CO₂ by hexaelectronic reduction (by hydrogenation or electro-reduction) is not favourable and even less economically viable. On the other hand, 2 e⁻ electroreduction is on track to become so.

An alternative to the preceding methods is based on the dismutation of formic acid into CO₂ and methanol as shown in FIG. 2 (Equation 3). In this method, the formic acid may be obtained from hydrogenation or more advantageously from 2e⁻ electro-reduction of CO₂. The CO₂ produced by the dismutation reaction may be converted back into formic acid using the same method.

Dismutation is a reaction wherein a chemical species, in this instance formic acid, acts both as an oxidant and a reducing agent. This means that the chemical species, initially present with a single degree of oxidation, will be found, after the reaction, in the form of two species, one oxidised, the other reduced.

In this case, the dismutation reaction enables the use of formic acid, which is liquid at normal temperature and pressure, both as a source of carbon for substances containing one carbon atom but also as a source of hydride to obtain methanol.

It should be noted first of all that the dismutation reaction of formic acid into methanol and CO₂ is in competition with two other more conventional formic acid breakdown reactions shown in FIG. 3 and which are:
dehydrogenation of formic acid into CO₂ and H₂ (Equation (4))
dehydration of formic acid into CO and H₂O (Equation (5))

The current prior art of catalysts capable of carrying out the dismutation reaction (3) rather than (4) and/or (5) are very limited in number. The latter work exclusively in homogenous phase and are all based on transition metal complexes.

The first research in the field was conducted in 2013 by Miller, Goldberg et al. [A. J. Miller, D. M. Heinekey, J. M. Mayer, K. I. Goldberg, Angew. Chem. Int. Ed. Engl. 2013, 52, 3981-3984]. The authors demonstrated that the iridium complex [Cp*Ir(bpy)(H₂O)](OTf)₂ (Cp*=pentamethylcyclopentadienyl, bpy=2,2′-bipyridine) makes it possible to obtain methanol yields of up to 12%. This figure represents the selectivity of the reaction for the formation of methanol according to equation (3). This also means that 88% of the C—H bonds in H—COOH end with hydrogen according to equation (4) and not with methanol. This 12% selectivity is obtained at 60° C. in the presence of formic acid in aqueous solution (C=12 M). The conversion of formic acid in this case is merely 3%.

More recently, Cantat et al. [S. Savourey, G. Lefevre, J. C. Berthet, P. Thuery, C. Genre. T. Cantat, Angew. Chem. Int. Ed. Engl. 2014, 53, 10466-10470] demonstrated that by using a ruthenium complex [Ru(COD)(methylallyl)₂] (COD=cyclooctadiene) combined with the tridentate ligand “triphos” CH₃C(CH₂PPh₂)₃ in THF at 150° C., the selectivity (which is equivalent herein to the methanol yield) may attain 26.7%. Under the same conditions and in the presence of an acidic additive (methane sulphonic acid or MSA), the yield exceeds 50%. The latter result represents at the present time the highest methanol yield obtained by formic acid dismutation.

Finally, Parkin et al. demonstrated in 2015 [M. C. Neary, G. Parkin, Chem. Sci. 2015, 6, 1859-1865] that molybdenum compounds are capable of carrying out the formic acid dismutation reaction. More specifically, the complex CpMo(CO)₃H (Cp=cyclopentadienyl) is the most effective of the complexes evaluated and 21% selectivity is reported at 100° C. in benzene.

This prior art reveals that the formic acid dismutation reaction is, at the present time, only enabled by the use of metals, most often noble and non-selective. At the present time, there are no non-metal compounds suitable for carrying out this transformation.

Therefore, there is a genuine need for a non-metal compound enabling metal-free dismutation of formic acid.

In particular, there is a genuine need for a compound which enables metal-free dismutation of formic acid, and which does not contain:
alkaline earth metals from Group IIA of the Periodic Table of the Elements (such as magnesium and calcium);
transition metals from Group IB to VIIIB of the Periodic Table of the Elements (such as nickel, iron, cobalt, zinc, copper, rhodium, ruthenium, platinum, palladium, iridium);
rare earths, the atomic number whereof is between 57 and 71 (such as lanthanum, cerium, praseodymium, neodymium); or
actinides, the atomic number whereof is between 89 and 103 (such as thorium, uranium).

There is also a genuine need for a non-metal compound as defined above, enabling the dismutation of formic acid into methanol, which is effective (capable of increasing the conversion rate of formic acid into methanol even when it is present in small quantities), selective (promoting the production of the substance sought with respect to secondary products), inexpensive and/or has a low toxicity compared to known metal compounds for the dismutation reaction of formic acid into methanol.

The present invention specifically has the aim of meeting these needs by providing a method for preparing methoxyboranes according to formula (I)

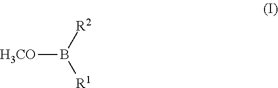

wherein
R¹ and R², independently of one another, are chosen in the group formed by a hydroxyl group, an alkoxy group, an alkyl group, an alkenyl group, an alkynyl group, an aryl group, a heteroaryl group, a heterocyclic group, a halogen group, a silyl group, a siloxy group, a phosphino group, and an amino group, said alkyl, alkenyl, alkynyl, alkoxy, silyl, siloxy, aryl, heteroaryl, heterocyclic, phosphino and amino groups being optionally substituted; or
R¹ and R² taken together with the boron atom to which they are bound, form an optionally substituted heterocycle;

by dismutation of formic acid or of at least one of the derivatives thereof having the formula HCO₂M wherein M is chosen in the group formed by Na⁺, K⁺, Li⁺, Cs⁺, NH₄⁺, triethylammonium (HNEt₃⁺), tetraphenylphosphonium (PPh₄⁺), tetramethylammonium (NMe₄⁺), tetraethylammonium (NEt₄⁺), tetrabutylammonium (NBu₄⁺) and tetraphenylammonium (NPh₄⁺), or of a mixture of formic acid and of at least one of the derivatives thereof.

in the presence
of an organoborane according to formula (II)

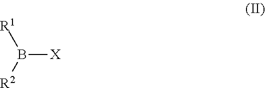

wherein
R¹ and R² are as defined for the methoxyborane compounds according to formula (I);
X is chosen in the group formed by a hydrogen atom, a halogen atom, a carboxylate group, a sulphonate group, a hydroxyl, an alkoxy group, an alkyl group, an alkenyl group, an alkynyl group, an aryl group, a heteroaryl group, a heterocyclic group, a silyl group, a siloxy group, a phosphino group, an amino group, said alkyl, alkenyl, alkynyl, alkoxy, silyl, siloxy, aryl, heteroaryl, heterocyclic, phosphino and amino groups being optionally substituted; and optionally
of an organic or inorganic base.

The organoborane according to formula (II) thereby enables the metal-free dismutation of formic acid or of one of the derivatives thereof.

The methoxy borane according to formula (I) may subsequently undergo hydrolysis or protonolysis to supply methanol.

The hydrolysis or protonolysis step makes it possible to convert the methoxyborane according to formula (I) into free methanol CH₃OH, optionally by regenerating the organoboranes according to formula (II).

In fact, once the methoxyboranes have been obtained, two options can be envisaged to form free methanol: by merely reacting with water (hydrolysis) or with a stronger acid (protonolysis) such as for example formic acid, acetic acid or hydrochloric acid.

The organoboranes according to formula (II) obtained after the methoxyborane protonolysis or hydrolysis step may be reused to produce the methoxyboranes using the inventive method.

The method for producing methanol involving a dismutation step of formic acid, one of the derivatives thereof, or the mixture thereof as defined above, has the following advantages:
Formic acid is liquid under normal temperature (20±5° C.) and pressure (1 atmosphere or 1.01325 bar) conditions, which makes it easy to transport with lower costs (and therefore risks) than the transportation of gas such as hydrogen. This is particularly advantageous when the production of formic acid and the dismutation are carried out in two separate locations. This step therefore consists of storing hydrogen and CO₂ in the form of formic acid.
Formic acid is non-toxic in dilute solution (concentration less than 85% by mass in water) and is hence a mild reagent.
In the present method, the dismutation reaction is carried out using organic boron derivatives, therefore free from metal, which avoids the drawbacks of existing metal derivatives, particularly a problematic cost and toxicity.
The organoboranes used according to formula (II) are commercial, or readily prepared by those skilled in the art using inexpensive commercial reagents. On the other hand, when metals are used, the ligands used for stabilising same are expensive and the preparation thereof generally requires multiple synthesis steps.

According to the present invention, an “alkyl” group denotes a linear, branched or cyclic, saturated, optionally substituted carbon-based radical, comprising 1 to 12 carbon atoms. By way of saturated, linear or branched alkyl, mention may be made for example of methyl, ethyl, propyl, butyl, pentyl, hexyl, octyl, nonyl, decyl, undecyl, dodecanyl radicals and the branched isomers thereof. By way of cyclic alkyl, mention may be made of cyclopropyl, cyclobutyl, cyclopentyl, cyclohexyl, cycloheptyl, cyclooctyl, bicylco[2,1,1]hexyl, bicyclo[2,2,1] heptyl radicals. The alkyl may comprise for example 1 to 8 carbon atoms.

The term “alkenyl” or “alkynyl” denotes an optionally substituted, unsaturated linear, branched or cyclic carbon-based radical, said unsaturated carbon-based radical comprising 2 to 12 carbon atoms comprising at least a double (alkenyl) or a triple bond (alkynyl). As such, mention may be made, for example, of ethylenyl, propylenyl, butenyl, pentenyl, hexenyl, acetylenyl, propynyl, butynyl, pentynyl, hexynyl radicals and the branched isomers thereof. By way of cyclic alkenyl, mention may be made for example of cyclopentenyl, cyclohbexenyl. The alkenyl and alkynyl groups may comprise for example 2 to 8 carbon atoms.

The alkyl, alkenyl, alkynyl groups may be optionally substituted by one or a plurality of hydroxyl groups; one or a plurality of alkoxy groups; one or a plurality of halogen atoms chosen from fluorine, chlorine, bromine or iodine atoms; one or a plurality of nitro groups (—NO₂); one or a plurality of nitrile groups (—CN); one or a plurality of aryl groups, with the alkoxy and aryl groups as defined within the scope of the present invention.

The term “aryl” denotes generally a cyclic aromatic substituent comprising 6 to 20 carbon atoms. Within the scope of the invention, the aryl group may be mono- or polycyclic. By way of indication, mention may be made of the phenyl, benzyl and naphthyl groups. The aryl group may be optionally substituted by one or a plurality of hydroxyl groups, one or a plurality of alkoxy groups, one or a plurality of siloxy groups, one or a plurality of halogen atoms chosen from fluorine, chlorine, bromine or iodine atoms, one or a plurality of nitro groups (—NO₂); one or a plurality of nitrile groups (—CN), one or a plurality of alkyl groups, with the alkoxy, alkyl and siloxy groups as defined within the scope of the present invention. The aryl group may comprise for example 6 to 10 carbon atoms.

The term “heteroaryl” denotes generally a mono- or polycyclic aromatic substituent comprising 5 to 12 members of which at least 2 carbon atoms, and at least one heteroatom chosen from nitrogen, oxygen, boron, silicon, phosphorus or sulphur. The heteroaryl may be mono- or poly-cyclic. By way of indication, mention may be made of the furyl, benzofuranyl, pyrrolyl, indolyl, isoindolyl, azaindolyl, thiophenyl, benzothiophenyl, pyridyl, quinolinyl, isoquinolyl, imidazolyl, benzimidazolyl, pyrazolyl, oxazolyl, isoxazolyl, benzoxazolyl, thiazolyl, benzothiazolyl, isothiazolyl, pyridazinyl, pyrimidilyl, pyrazinyl, triazinyl, cinnolinyl, phtalazinyl, quinazolinyl groups. The heteroaryl group may be optionally substituted by one or a plurality of hydroxyl groups, one or a plurality of alkoxy groups, one or a plurality of halogen atoms chosen from fluorine, chlorine, bromine or iodine atoms, one or a plurality of nitro groups (—NO₂), one or a plurality of nitrile groups (—CN), one or a plurality of aryl groups, one or a plurality of alkyl groups, with the alkyl, alkoxy and aryl groups as defined within the scope of the present invention.

The term “heterocycle or heterocyclic” denotes generally a mono- or polycyclic substituent, comprising 5 to 10 members, saturated or unsaturated, containing I to 4 heteroatoms chosen independently of one another, from nitrogen, oxygen, boron, silicon, phosphorus or sulphur. By way of indication, mention may be made of borolane, borole, borinane, 9-borabicyclo[3.3.1]nonane (9-BBN), 1,3,2-benzodioxaborole (catecholborane or catBH), pinacholborane (pinBH), the substituents morpholinyl, piperidinyl, piperazinyl, pyrrolidinyl, imidazolidinyl, imidazolinyl, pyrazolidinyl, tetrahydrofuranyl, tetrahydropyranyl, thianyl, oxazolidinyl, isoxazolidinyl, thiazolidinyl, isothiazolidinyl. The heterocycle may be optionally substituted by one or a plurality of hydroxyl groups, one or a plurality of alkoxy groups, one or a plurality of aryl groups, one or a plurality of halogen atoms chosen from fluorine, chlorine, bromine or iodine atoms, one or a plurality of nitro groups (—NO₂), one or a plurality of nitrile groups (—CN), one or a plurality of aryl groups, one or a plurality of alkyl groups, with the alkyl, alkoxy and aryl groups as defined within the scope of the present invention.

The term “alkoxy” denotes an alkyl group, as defined above, bound with an oxygen atoms (—O-alkyl).

The term “amino” group denotes a group having the formula —NR³R⁴, wherein:
R³ and R⁴ represent, independently of one another, an alkyl group, an alkenyl group, an alkynyl group, an aryl group, a heteroaryl group, a heterocyclic group, a silyl group, a siloxy group, with the alkyl, alkenyl, alkynyl, aryl, heteroaryl, heterocyclic, silyl, siloxy groups, as defined within the scope of the present invention; or
R³ and R⁴, taken together with the nitrogen atom to which they are bound, form a heterocycle optionally substituted by one or a plurality of hydroxyl groups; one or a plurality of alkyl groups; one or a plurality of alkoxy groups; one or a plurality of halogen atoms chosen from fluorine, chlorine, bromine or iodine atoms; one or a plurality of nitro groups (—NO₂); one or a plurality of nitrile groups (—CN); one or a plurality of aryl groups; with the alkyl, alkoxy and aryl groups as defined within the scope of the present invention.

The amino group may be, for example, a substituent chosen in the group formed by -NEt₂, piperidinyl, morpholinyl, methylaniline (C₆H₅N(CH₃)—), and triazabicyclodecenyl (TBD).

The term “phosphino” group denotes a group having the formula —PR⁵R⁶, wherein:
R⁵ and R⁶ represent, independently of one another, an alkyl group, an alkenyl group, an alkynyl group, an aryl group, a heteroaryl group, a heterocyclic group, a silyl group, a siloxy group, with the alkyl, alkenyl, alkynyl, aryl, heteroaryl, heterocyclic, silyl, siloxy groups, as defined within the scope of the present invention; or
R⁵ and R⁶, taken together with the phosphorus atom to which they are bound, form a heterocycle optionally substituted by one or a plurality of hydroxyl groups; one or a plurality of alkyl groups; one or a plurality of alkoxy groups; one or a plurality of halogen atoms chosen from fluorine, chlorine, bromine or iodine atoms; one or a plurality of nitro groups (—NO₂); one or a plurality of nitrile groups (—CN); one or a plurality of aryl groups; with the alkyl, alkoxy and aryl groups as defined within the scope of the present invention.

The phosphino group may be, for example, a substituent chosen in the group formed by PCy₂ where Cy=cyclohexyl, and PPh₂ where Ph=phenyl.

The term halogen atom denotes an atom chosen from fluorine, chlorine, bromine and iodine atoms.

The term “sulphonate” denotes a group having the formula —OSO₂R⁷, wherein:
R⁷ is chosen from: a methyl group (CH₃) a trifluoromethyl group (CF₃), a toluene group (p-CH₃C₆H₄) or a benzene group (C₆H₅).

The term “carboxylate” denotes a group having the formula —OC(O)R⁸, wherein R⁸ is chosen from a hydrogen atom, an alkyl group, an alkenyl group, an alkynyl group, an aryl group, a heteroaryl group, a heterocyclic group, a silyl group, a siloxy group, with the alkyl, alkenyl, alkynyl, aryl, heteroaryl, heterocyclic groups, as defined within the scope to the present invention.

The term “silyl” group denotes a group having the formula [—Si(Y)₃] wherein each Y, independently of one another, is chosen from a hydrogen atom; one or a plurality of halogen atoms chosen from fluorine, chlorine, bromine or iodine atoms; one or a plurality of alkyl groups; one or a plurality of alkoxy groups; one or a plurality of amino groups; one or a plurality of aryl groups; one or a plurality of siloxy groups; with the alkyl, alkoxy, aryl and siloxy groups as defined within the scope of the present invention. As such, mention may be made, for example, of trimethylsilyl (TMS), triethylsilyl (TES), tert-butyldiphenylsilyl (TBDPS), tert-butyldimethylsilyl (TBS;TBDMS), triisopropylsilyl (TIPS), tri(trimethylsilyl)silyl or ((CH₃)₃Si)₃Si— (TTMS), tri(tert-butyl)silyl or ((CH₃)₃C)₃Si—.

The term “siloxy” denotes a silyl group, as defined above, bound by an oxygen atom (—O—Si(Y)₃) where Y is as defined above. As such, mention may be made, for example, of trimethylsiloxy, triethylsiloxy, tert-butyldiphenylsiloxy.

In the context of the invention, “protonolysis” denotes the splitting of a chemical bond by Brønsted acids. Hydrolysis denotes the splitting of a chemical bond by water.

According to a first alternative embodiment of the invention, in the methoxyborane according to formula (I) and the organoborane according to formula (II), R¹ and R², independently of one another, are chosen in the group formed by an alkyl group comprising 1 to 12, preferably 1 to 8 carbon atoms; an aryl comprising 6 to 20, preferably 6 to 10 carbon atoms, said alkyl and aryl groups being optionally substituted. Preferably, R¹ and R², independently of one another, are chosen in the group formed by methyl, ethyl, propyl, butyl, pentyl, hexyl, heptyl and octyl and the branched isomers thereof, cyclohexyl, phenyl, benzyl. Even more preferentially, R¹ and R², independently of one another, are chosen in the group formed by butyl and the branched isomers thereof, and cyclohexyl.

According to a second alternative embodiment of the invention, in the methoxyborane according to formula (I) and the organoborane according to formula (II), R¹ and R² taken together with the boron atom to which they are bound, form a heterocycle comprising 5 to 10 members, said heterocycle being optionally substituted. Preferably, R¹ and R² taken together with the boron atom to which they are bound, form a 9-borabicyclo[3.3.1]nonane (9-BBN), a 1,3,2-benzodioxaborole (catecholborane or catBH), or a pinacholborane (pinBH).

In all the alternative embodiments and all the preferred embodiments, X may for example be chosen in the group formed by a hydrogen atom; a carboxylate group having the formula —OCOR⁸ wherein R⁸ is chosen from a hydrogen atom, an alkyl group comprising 1 to 12, preferably 1 to 8 carbon atoms; a halogen atom; an alkyl group comprising 1 to 12, preferably 1 to 8 carbon atoms; a sulphonate group having the formula —OSO₂R⁷, wherein R⁷ is chosen from a methyl group (CH₃), a trifluoromethyl group (CF₃), a toluene group (p-CH₃C₆H₄) or a benzene group (C₆H₅). In particular, X is chosen in the group formed by a hydrogen atom; a carboxylate group having the formula —OCOR⁸ wherein R⁸═H; a chlorine atom; a boron atom; an alkyl group chosen in the group formed by methyl, ethyl, propyl, butyl, pentyl, hexyl and the branched isomers thereof; a sulphonate group having the formula —OSO₂R⁷, wherein R⁷ is chosen from a methyl group, a trifluoromethyl group (CF₃), a toluene group (p-CH₃C₆H₄) or a benzene group (C₆H₅).

Preferably, the organoborane according to formula (II) may be chosen in the group formed by tributylborane, dicyclohexylborane, iododicyclohexylborane, dibutylborane methanesulphonate (n-Bu₂BOSO₃Me), B-iodo-9-borabicyclo[3.3.1]nonane, the dimer of 9-borabicyclo[3.3.1]nonane, B-benzyl-9-borabicyclo[3.3.1]nonane, Me-TBD-BBN⁺I⁻.

The dismutation of formic acid, of at least one of the derivatives thereof, or of the mixture thereof as described above, may take place advantageously in the presence of an organic or inorganic base, preferably organic.

The organic base may be chosen from:
nitrogen-containing organic bases which are advantageously tertiary amines chosen in the group formed by, for example, triethylamine, trimethylamine, N-diisopropylethylamine (DIPEA), diethylisopropylamine (DIEA), 7-Methyl-1,5,7-triazabicyclo[4.4.0]dec-5-ene (MTBD), 1,8-diazabicyclo[5.4.0]undec-7-ene (DBU), 1,4-diazabicyclo[2.2.2]octane (DABCO), and N-methylpiperidine;
phosphorus-containing organic bases optionally being alkyl or aryl phosphines chosen in the group formed by, for example, triphenylphosphine, 2,2′-bis(diphenylphosphino)-1,1′-binaphthyl (BINAP), triisopropylphosphine, 1,2-bis(diphenylphosphino)ethane (dppe), tricyclohexylphosphine (PCy₃); aza-phosphines chosen in the group formed, for example, by 2,8,9-triisopropyl-2,5,8,9-tetraaza-1-phosphabicyclo[3.3.3]undecane (BV^{Me}) and 2,8,9-triisobutyl-2,5,8,9-tetraaza-1-phosphabicyclo[3.3.3]undecane (BV^{iBu});
carbon-containing bases for which protonation takes place on a carbon atom, chosen advantageously from the N-heterocyclic carbenes derived from an imidazolium salt, said carbenes being, for example, chosen in the group formed by the salts of 1,3-bis(2,6-diisopropylphenyl)-1H-imidazol-3-ium (also referred to as IPr), 1,3-bis(2,6-diisopropylphenyl)-4,5-dihydro-1H-imidazol-3-ium (also referred to as s-IPr), 1,3-bis(2,4,6-trimethylphenyl)-1H-imidazol-3-ium (also referred to as IMes), 1,3-bis(2,4,6-trimethylphenyl)-4,5-dihydro-1H-imidazol-3-ium (also s-IMes), 4,5-dichloro-1,3-bis(2,6-diisopropylphenyl)-1H-imidazol-3-ium (also referred to as Cl₂-IPr), 1,3-di-tert-butyl-1H-imidazol-3-ium (also referred to as ItBu), and 1,3-di-tert-butyl-4,5-dihydro-1H-imidazol-3-ium (also referred to as s-ItBu), said salts being in the form of chloride or tetraphenylborate salts, for example.

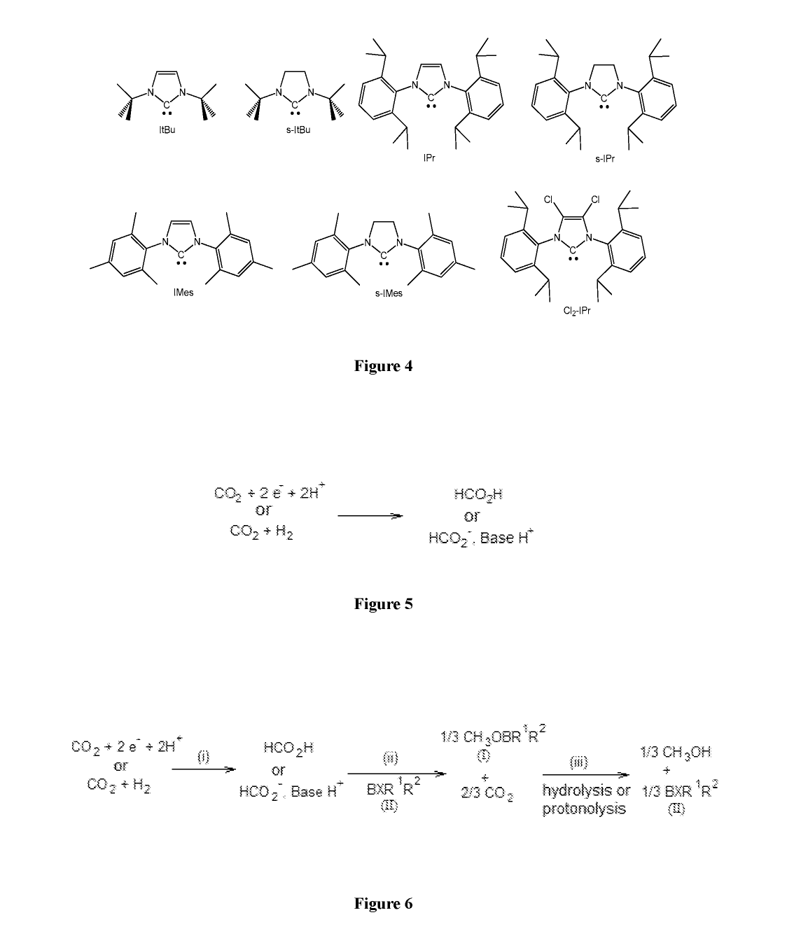

Examples of N-heterocyclic carbenes are represented in FIG. 4.

Preferably, the organic base is a nitrogen-containing organic base chosen in the group formed by triethylamine, trimethylamine, N-diisopropylethylamine (DIPEA), diethylisopropylamine (DIEA), 7-Methyl-1,5,7-triazabicyclo[4.4.0]dec-5-ene (MTBD), 1,8-diazabicyclo[5.4.0]undec-7-ene (DBU), 1,4-diazabicyclo[2.2.2]octane (DABCO), and N-methylpiperidine.

When the base is an inorganic base, it consists of a mineral compound having basic properties according to Brønsted. As such, mention may be made, for example, of NaH, KH, NaHCO₃, Na₂CO₃.

When starting with a derivative of formic acid having the formula HCOOM (for example HCOONa), and it is reacted on the organoborane according to formula (II), it is not generally necessary to introduce a base into the medium as the formic acid has already been deprotonated to obtain this derivative. The same applies if X=H and a mixture of pure formic acid and HCOONa (1:1), for example, is used.

If deemed necessary, an additive may also be used to solubilise the starting reagents. The term additive, according to the invention, denotes any compound capable of improving the solubility of the reagents involved in the dismutation of formic acid, one of the derivatives thereof or a mixture of formic acid and at least one of the derivatives thereof. These additives are introduced when formic acid derivatives such as the alkaline salts of the anion formiate are used (for example HCOONa or HCOOK) or when an inorganic base is used. The dismutation of formic acid, or of at least one of the derivatives thereof or of a mixture of formic acid and at least one of the derivatives thereof as defined above, may further take place in the presence of an additive. These additives may be chosen, for example, from:
crown ethers chosen in the group formed by 12-crown-4, 15-crown-5, 18-crown-6, dibenzo-18-crown-6, benzo-18-crown-6, benzo-15-crown-5, and dibenzo-15-crown-5;
aza-crowns chosen in the group formed by 1,4,7,10-tetraazacyclododecane (cyclene), 1,4,7,10,13,16-hexaazacyclooctadecane (hexacyclene), and diaza-18-crown-6;
crown thioethers chosen in the group formed by 1,5,9,13-tetrathiacyclohexadecane (16-Ane-S₄), and 1,4,7,10,13,16-hexathiacyclooctadecane (18-Ane-S₆).

The organoboranes according to formula (II) may, if applicable, be fixed on heterogenous substrates so as to ensure the ready separation and/or recycling thereof. Said heterogenous substrates may be chosen from substrates based on silica gel or plastic polymers such as, for example, polystyrene; carbon-based substrates chosen from carbon nanotubes; silica carbide; or alumina.

The formic acid or one of the derivatives thereof as defined above may be prepared by any method known to those skilled in the art. In the present disclosure, methods known to be able to carry out this step have been described. Preferably, the formic acid is prepared by 2 e⁻ electro-reduction or catalytic hydrogenation of CO₂ as shown in FIG. 5.

The derivatives of formic acid are generally obtained therefrom. However, there is a large number of methods, and particularly the hydrogenation of CO₂ which are suitable for obtaining formic acid derivatives directly. Obtaining protonated formic acid requires an additional step, typically by adding a strong acid. The references describing the preparation of formic acid derivatives are those cited above describing the hydrogenation of CO₂. The formic acid derivatives may also be prepared very readily by any method well-known to those skilled in the art, that is to say, by reacting a base (for example triethylamine) with formic acid. It should be noted that the base used must have a pKa greater than that of formic acid, i.e. greater than 3.7.

The formic acid may also be generated from one of the derivatives thereof by reacting with hydrochloric acid (salt reprotonation) or any other strong acid in water.

As already stated, catalytic hydrogenation of CO₂ requires the use of a base to shift the equilibrium towards the formation of the formiate salt. As such, when catalytic hydrogenation of CO₂ is chosen and, in the method according to the invention, the dismutation requires the presence of a base, both reactions may be carried out in succession in the same reactor.

If 2 e⁻ electro-reduction of CO₂ is chosen to prepare the formic acid and the dismutation requires the presence of a base, both reactions may be carried out in succession in the same reactor. The base should then be added.

The 2e⁻ reduction of CO₂ is preferentially carried out according to the most selective methods known to date. For example, catalytic hydrogenation of CO₂ may be carried out according to the protocol of Nozaki et al. [R. Tanaka, M. Yamashita, K. Nozaki, J. Am. Chem. Soc. 2009, 131, 14168-14169] and makes it possible to retrieve potassium formiate. The electro-reduction may for its part be conducted according to the conditions of Shibata et al. or of Hori et al. [Y. Hori, H. Wakebe, T. Tsukamoto, O. Koga, Electrochim. Acta 1994, 39, 1833-1839; N. Furuya, T. Yamazaki, M. Shibata. J. Electroanal. Chem. 1997, 431, 39-41]

It should be noted that electro-reductions are dependent on a large number of parameters such as the type of reactor, the electrodes, the electrolyte or the electro-catalyst used. These concepts and the various systems suitable for obtaining formic acid or one of the derivatives thereof are discussed in the reviews by Olah et al., Leung, Xuan et al. or Kenis et al. [A. Goeppert, M. Czaun, J. P. Jones, G. K. Surya Prakash, G. A. Olah, Chem. Soc. Rev. 2014, 43, 7995-8048; H.-R. M. Jhong, S. Ma, P. J. A. Kenis, Curr. Opin. Chem. Eng. 2013, 2, 191-199; X. Lu, D. Y. C. Leung, H. Wang, M. K. H. Leung, J. Xuan, Chem Electro Chem 2014, 1, 836-849]. The formic acid or one of the derivatives formed (sodium, potassium, lithium, caesium, ammonium formiate, for example) formed may subsequently be used in the dismutation step.

The invention relates to a method for producing methanol characterised in that it comprises

(A) a step for preparing a methoxyborane according to formula (I) by dismutation of formic acid, or of at least one of the derivatives thereof having the formula HCO₂M wherein M is chosen in the group formed by Na⁺, K⁺, Li⁺, Cs⁺, NH₄⁺, triethylammonium (HNEt₃⁺), tetraphenylphosphonium (PPh₄⁺), tetramethylammonium (NMe₄⁺), tetraethylammonium (NEt₄⁺), tetrabutylammonium (NBu₄⁺), and tetraphenylammonium (NPh₄⁺), or of a mixture of formic acid and at least one of the derivatives thereof,

in the presence

of an organoborane according to formula (II); and optionally

of an organic or inorganic base,

according to the method of the invention;

(B) a step for the hydrolysis or protonolysis of the methoxyborane according to formula (I) obtained following the dismutation step (A) into methanol.

Prior to the step (A), the formic acid or derivatives thereof as defined above are prepared, preferably, by 2e⁻ reduction of CO₂, or by catalytic hydrogenation of CO₂.

The invention also relates to a method for producing methanol characterised in that it comprises

(i) a step for preparing formic acid or the derivatives thereof having the formula HCO₂M wherein M is chosen in the group formed by Na⁺, K⁺, Li⁺, Cs⁺, NH₄⁺, triethylammonium (HNEt₃⁺), tetraphenylphosphonium (PPh₄⁺), tetramethylammonium (NMe₄⁺), tetraethylammonium (NEt₄⁺), tetrabutylammonium (NBu₄⁺), and tetraphenylammonium (NPh₄⁺), by 2e⁻ reduction of CO₂, or by catalytic hydrogenation of CO₂;

(ii) a step for preparing a methoxyborane according to formula (I) by dismutation of formic acid or of at least one of the derivatives thereof, or of a mixture of formic acid and at least one of the derivatives thereof, according to the method of the invention; and

(iii) a step for the hydrolysis or protonolysis of the methoxyborane according to formula (I) obtained following the dismutation step (ii) into methanol.

This method is represented in FIG. 6. The gases generated in this method (essentially CO₂ and lesser quantities of H₂) may be retrieved and reused in step (i) of the method. Similarly, the organoborane according to formula (II) generated in this method may be retrieved after the protonolysis step and reused in the dismutation step (ii).

This method for producing methanol is a particular case of the method for producing methanol described above. For this reason, steps (ii) and (iii) of this method for producing methanol are identical to steps (A) and (B) of the method for producing methanol described above. As such, everything described in respect in particular of the operating conditions, advantages and specificities for steps (ii) and (iii) of this method applies in the same way to steps (A) and (B) of the method described above, and conversely. Similarly, everything described in respect in particular of the operating conditions, advantages and specificities relating to the dismutation reaction applies to the method for preparing a methoxyborane according to formula (I) and to both methods for producing methanol described.

Step (ii) or (A) consists of reacting:
formic acid or at least one of the derivatives thereof previously obtained in step (i) or prior to step (A), or a mixture of formic acid and at least one of the derivatives thereof;
an organoborane according to formula (II);
optionally a base, preferably organic, as specified above. When an inorganic base is used, it is possible that an additive such as those described above may be required so as to solubilise all the reagents.

The various reagents used in the methods according to the invention (organoboranes according to formula (II), formic acid or derivatives thereof, base, additive, acids, etc.) are, in general, commercial compounds or may be prepared by any method known to those skilled in the art.

The quantity of organoborane according to formula (II) is 0.05 to 1 molar equivalent, preferably 0.5 to 1 molar equivalent, inclusive, with respect to formic acid or to the derivative(s) thereof or to a mixture of formic acid and at least one of the derivatives thereof.

When a base is used, the quantity of 0.05 to 3 molar equivalents preferably 0.5 to 1.5 molar equivalents inclusive, with respect to formic acid or to the derivative(s) thereof or to a mixture of formic acid and at least one of the derivatives thereof.

When an additive is used, the quantity of additive is 1 to 2 molar equivalents preferably 1 to 1.5 molar equivalents inclusive, with respect to the formic acid derivative(s) or to a mixture of formic acid and at least one of the derivatives thereof.

The number of equivalents of the organoborane according to formula (II), the base and the additive are in fact calculated with respect to the number of equivalents of HCOO⁻. For example, in the case of a mixture of formic acid with at least one of the derivatives thereof such as for example an HCOOH/HCOONa (1:1) mixture, the number of formal HCOO⁻ equivalents is 2.

The dismutation reaction generates a gas pressure resulting from the formation of carbon dioxide and optionally dihydrogen. The reaction may then take place under pressure of the gases formed or under atmospheric pressure by collecting the gases, for example in a burette. The gas(es) may be reused to prepare the formic acid or one of the derivatives thereof for example in step (i) of the method, that is to say to carry out the 2e⁻ reduction of CO₂.

The temperature of the dismutation reaction may be between 25 and 150° C., preferably between 25 and 130° C., inclusive.

The reaction time is dependent on the formic acid conversion rate. The reaction may be carried out for a time of 5 minutes to 200 hours, preferably 10 minutes to 48 hours, inclusive.

The dismutation step (ii) or (A) may be carried out in pure formic acid, in one of the derivatives of formic acid if the latter is liquid or using one or a mixture of at least two solvents. The solvent may be chosen in the group formed by:
water;
alcohols, for example, ethanol or ethylene glycol;
ethers, for example, diethyl ether, THF, diglyme, 1,4-dioxane;
hydrocarbides, for example, benzene, or toluene;
nitrogen-based solvents, for example, pyridine, or acetonitrile;
sulphoxides, for example, dimethyl sulphoxide;
alkyl halides, for example, chloroform, or methylene chloride; and
a supercritical fluid, for example, supercritical CO₂.

Preferably, the dismutation reaction is conducted in an aprotic polar solvent chosen from THF or acetonitrile. More preferentially, the solvent is acetonitrile.

The hydrolysis or protonolysis step (iii) or (B) makes it possible to convert the methoxyborane according to formula (I) into free methanol CH₃OH, optionally by regenerating the organoboranes according to formula (II).

The organoboranes according to formula (II) obtained after the step for the protonolysis or hydrolysis of the methoxyboranes may be reused to produce the methoxyboranes by means of the inventive method.

The hydrolysis or protonolysis step may be carried out with any acid known to those skilled in the art, for example, formic acid, acetic acid or hydrochloric acid.

In fact, once the methoxyboranes have been obtained, two options can be envisaged to form free methanol: by merely reacting with water (hydrolysis) or with a stronger acid (protonolysis) such as for example formic acid, acetic acid or anhydrous hydrochloric acid.

The methanol obtained by means of the methods according to the invention may be suitable for use in any method using methanol, particularly any method intending to use methanol as a fuel or additive in combustion engines, as a fuel in direct methanol fuel cells, as a means for storing hydrogen (E. Alberico, M. Nielsen, Chem. Comm. 2015, 51, 6714-6725), as a reagent for fine chemistry or as a reagent for heavy chemistry.

As such, the invention further relates to the use of methoxyboranes according to formula (I) obtained by means of the method according to the invention,
for producing methanol,
as catalysts in allylation reactions,
as reagents in Suzuki coupling reactions,
as a reagent for fine chemistry or for heavy chemistry.

The methanol produced by hydrolysis or protonolysis of the methoxyboranes formed by the inventive method may be used as a fuel in direct methanol fuel cells, as a means for storing hydrogen, as a reagent for fine chemistry.

Further advantages and features of the present invention will emerge on reading the examples hereinafter given by way of illustration and not limitation and the appended figures:

FIG. 1 represents the hexaelectronic (with 6 electrons i.e. 3 hydrides) reduction of CO₂ into methanol by catalytic hydrogenation (Equation (1)), and the hexaelectronic electro-reduction of CO₂ into methanol (Equation (2));

FIG. 2 represents the dismutation of formic acid into CO₂ and methanol;

FIG. 3 represents two formic acid breakdown reactions which are in competition with the dismutation reaction of formic acid into methanol and CO₂. These two reactions are: dehydrogenation of formic acid into CO₂ and H₂ (Equation (4)) and dehydration of formic acid into CO and H₂O (Equation (5));

FIG. 4 represents examples of N-heterocyclic carbenes;

FIG. 5 represents the preparation of formic acid by 2 e⁻ electro-reduction or catalytic hydrogenation of CO₂;

FIG. 6 represents the method for producing methanol comprising (i) a step for preparing formic acid or one of the derivatives thereof by 2e reduction of CO₂, or by catalytic hydrogenation of CO₂; (ii) a step for preparing a methoxyborane according to formula (I) by dismutation of formic acid or at least of one of the derivatives thereof, or of a mixture of formic acid and at least one of the derivatives thereof; and (iii) a step for the hydrolysis or protonolysis of the methoxyborane according to formula (I) obtained following the dismutation step (ii) into methanol;

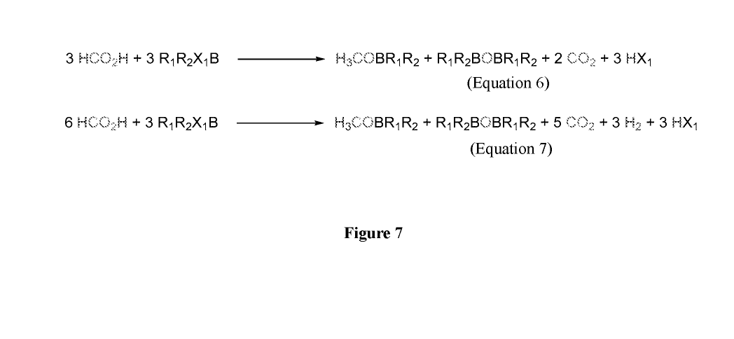

FIG. 7 represents the reactions involved in calculating the yields for obtaining methoxyboranes.

### EXAMPLES

A set of results is presented hereinafter, giving examples of dismutation of formic acid into methoxyborane according to formula (I) and CO₂ in the presence of an organoborane according to formula (II). The yields are obtained by ¹H NMR by integrating the signals of the methoxyborane according to formula (I) or the methanol obtained after hydrolysis or protonolysis of the methoxyborane according to formula (I) with respect to those of an internal standard (mesitylene). The yields are calculated by observing the stoichiometry of the dismutation reaction, that is to say that, at best, 3 moles of formic acid produce not more than maximum 1 mol of methanol (see equation 3 in FIG. 2).

$$ρ ( {MeOBR}^{1} {R}^{2} ) = \frac{3 \times n ( {MeOBR}^{1} {R}^{2} )}{{n}_{0} ( HCOOZ )}$$

n₀(MeOBR¹R²): quantity of methoxyborane substance determined by ¹H NMR with respect to mesitylene

n₀(HCOOZ)=n₀(HCOOH)+n₀(HCOOM): quantity of total substance in formic acid (HCOOH) and the derivatives thereof (HCOOM, M as defined above) introduced initially.

In our case, the use of organoboranes requires equations 6 and 7 (FIG. 7). The yields are nonetheless calculated on the basis of equation 3.

The hydrolysis yield being quantitative, the yields of methoxyboranes according to formula (I) and methanol are equal (FIG. 6).

The dismutation reaction of formic acid may be carried out according to the following experimental protocol:
1. In an inert atmosphere, in a glovebox, the organoborane according to formula (II), the formic acid or one of the derivatives thereof, or a mixture of formic acid and at least one of the derivatives thereof and optionally, the solvent and/or the base and/or the additive are introduced into a Schlenk tube which is subsequently sealed with a J. Young valve. The order of introduction of the reagents is of no importance.
2. The Schlenk is then heated to a temperature between 25 and 150° C. (preferentially >110° C.) until the complete conversion of the formic acid (from 5 minutes to 72 hours of reaction). The reaction is monitored by ¹H and/or ¹³C and/or ¹¹B proton NMR.
3. When the reaction is complete (which corresponds to the disappearance of the characteristic signals of the protons of the formiate H—COO⁻ in ¹H NMR), the pressure in the tube is released and the solvent as well as the volatile compounds are evaporated in a vacuum (10⁻² mbar).
   It should be noted that the gases generated by the reaction (essentially CO₂ and less quantities of H₂) may be retrieved and reused to generate formic acid by catalytic hydrogenation of CO₂.
4. The residue obtained after evaporating the volatile compounds is then hydrolysed or protonolysed respectively with H₂O or anhydrous HCl (in solution in diethyl ether) to release the free methanol which may be collected readily by distillation. In the case where HCl is used, it is also possible to retrieve chloroorganoboranes by distillation, the latter being suitable for reuse in the dismutation reaction according to the method.

### Example 1: Preparation of MeOBBN from BBNI, Formic Acid and Triethylamine

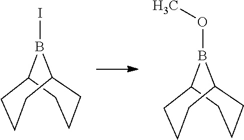

According to the general protocol described above, BBNI (commercial, 1 M in hexane, 100 μL, 0.1 mmol) is added to a mixture of formic acid (7.4 μL, 0.2 mmol, 2 equiv.) and triethylamine NEt₃ (27.8 μL, 0.2 mmol, 2 equiv.) in acetonitrile. The tube is then sealed and shaken vigorously to solubilise the borane adhering to the walls of the tube. Once the mixture is homogenous (<1 min), it is heated to 130° C. for 19 hours. The reaction mixture is then analysed by ¹H NMR and a 19% MeOBBN yield is obtained. The latter may then be hydrolysed to obtain free methanol. For this purpose, the tube is opened and 18 μL of water (1 mmol, 5 equiv.) is added. The tube is then stirred for 30 min at ambient temperature to quantitatively obtain methanol.

### Example 2: Preparation of MeOBBu₂ from Bu₂B-OTf, Formic Acid and Triethylamine

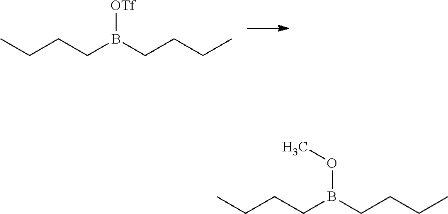 [SMILES: CCCCB(CCCC)OS(=O)(=O)C(F)(F)F ; CCCCB(CCCC)OC]

Using the same protocol as that of example 1, replacing BBNI by Bu₂B-OTf, a 15% yield of methanol borate MeOBBu₂ is obtained.

### Example 3: Preparation of MeOCy₂ from Cy₂BCl, Formic Acid and Triethylamine

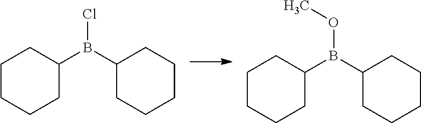

Using the same protocol as that of example 1, replacing BBNI by Cy₂BCl, a 17% yield of methanol borate McOBCy₂ is obtained.

### Example 4: Preparation of MeOBBu₂ from Bu₃B, Formic Acid and Triethylamine

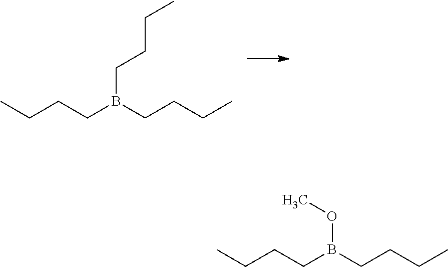

Using the same protocol as that of example 1, replacing BBNI by Bu₃B (commercial, 1 M in Et₂O), a 25% yield of methoxyborane MeOBBu₂ is obtained.

### Example 5: Preparation of MeOBBN from BBNH, Formic Acid and Triethylamine

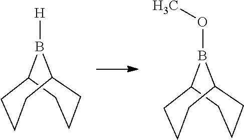

According to the general protocol described above, (BBNH)₂ (0.5 equiv.) is added to a mixture of formic acid (2 equiv.) and triethylamine NEt₃ (1 equiv.) in acetonitrile. The tube is then sealed and heated slightly (approx. 50° C.) to solubilise the solid reagents. A significant release of hydrogen is then observed, which once complete leaves a homogenous mixture (approx. 10 min). The latter is heated to 130° C. for 19 hours. The reaction mixture is then analysed by ¹H NMR and a 53% MeOBBN yield is obtained.

### Example 6: Preparation of MeOBBN from BBNH, Formic Acid and Diisopropylethylamine

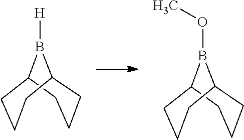

According to the general protocol described above, (BBNH)₂ (0.5 equiv.) is added to a mixture of formic acid (2 equiv.) and diisopropylethylamine i-Pr₂NEt (1 equiv.) in acetonitrile. The tube is then sealed and heated slightly (approx. 50° C.) to solubilise the solid reagents. A significant release of hydrogen is then observed, which once complete leaves a homogenous mixture (approx. 10 min). The latter is heated to 130° C. for 7 hours. The reaction mixture is then analysed by ¹H NMR and a 49% MeOBBN yield is obtained.

### Example 7: Preparation of MeOBBN from BBNH, Formic Acid and Sodium Formiate, in the Presence of the Crown Ether 15-C-5

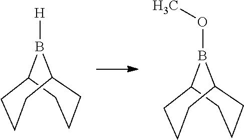

According to the general protocol described above, (BBNH)₂ (0.5 equiv.) is added to a mixture of formic acid (1 equiv.) and sodium formiate HCO₂Na (1 equiv.) in acetonitrile. The crown ether 15-C-5 (1 equiv.) is then added with a syringe and the tube heated slightly (approx. 50° C.) to solubilise the solid reagents. The latter is heated to 130° C. for 24 hours. The reaction mixture is then analysed by ¹H NMR and a 58% MeOBBN yield is obtained.

### Example 8: Preparation of MeOBCy₂ from Cy₂BH, Formic Acid and Triethylamine

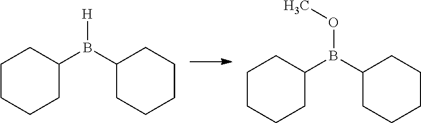

According to the general protocol described above, Cy₂BH (1 equiv.) is added to a mixture of formic acid (2 equiv.) and triethylamine NEt₃ (1 equiv.) in acetonitrile. The tube is then sealed and heated slightly (approx. 50° C.) to solubilise the solid reagents. A significant release of hydrogen is then observed, which once complete leaves a homogenous mixture (approx. 5 min). The latter is heated to 120° C. for 7 hours. The reaction mixture is then analysed by ¹H NMR and a 31% MeOBCy₂ yield is obtained.

### Example 9: Production of Methanol

The MeOBBN obtained in example 5 may then be hydrolysed to obtain free methanol:

Once the reaction is complete, the volatile compounds are evaporated in a vacuum (10⁻¹ to 10⁻² mbar), the solid residue obtained is then dissolved in THF and H₂O (5-10 equiv. with respect to the borane initially introduced) is added to the reaction mixture. The solution is stirred for 30 minutes to 1 hour at ambient temperature (20+5° C.). The volatile methanol is then retrieved in another Schlenk tube by transfer under reduced pressure. An aqueous methanol solution with a 50% yield is thereby obtained.

1. A method for preparing methoxyboranes according to formula (I) 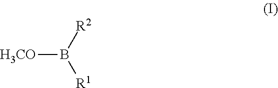
wherein
R¹ and R², independently of one another, are chosen in the group formed by a hydroxyl group, an alkoxy group, an alkyl group, an alkenyl group, an alkynyl group, an aryl group, a heteroaryl group, a heterocyclic group, a halogen group, a silyl group, a siloxy group, a phosphino group, and an amino group, said alkyl, alkenyl, alkynyl, alkoxy, silyl, siloxy, aryl, heteroaryl, heterocyclic, phosphino and amino groups being optionally substituted; or
R¹ and R² taken together with the boron atom to which they are bound, form an optionally substituted heterocycle;
by dismutation of formic acid or of at least one of the derivatives thereof having the formula HCO₂M wherein M is chosen in the group formed by Na⁺, K⁺, Li⁺, Cs⁺, NH₄⁺, triethylammonium (HNEt₃⁺), tetraphenylphosphonium (PPh₄⁺), tetramethylammonium (NMe₄⁺), tetraethylammonium (NEt₄⁺), tetrabutylammonium (NBu₄⁺) and tetraphenylammonium (NPh₄⁺), or of a mixture of formic acid and of at least one of the derivatives thereof,
in the presence
of an organoborane according to formula (II) 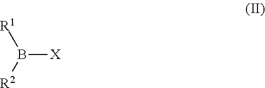
wherein
R¹ and R² are as defined for the methoxyborane compounds according to formula (I);
X is chosen in the group formed by a hydrogen atom, a halogen atom, a carboxylate group, a sulphonate group, a hydroxyl, an alkoxy group, an alkyl group, an alkenyl group, an alkynyl group, an aryl group, a heteroaryl group, a heterocyclic group, a silyl group, a siloxy group, a phosphino group, an amino group, said alkyl, alkenyl, alkynyl, alkoxy, silyl, siloxy, aryl, heteroaryl, heterocyclic, phosphino and amino groups being optionally substituted; and optionally
of an organic or inorganic base.

2. The method according to claim 1, wherein the formic acid and derivatives thereof are prepared by 2 e⁻ electro-reduction or catalytic hydrogenation of CO₂.

3. The method according to claim 1, wherein in the methoxyborane according to formula (I) and the organoborane according to formula (II), R¹ and R², independently of one another, are chosen in the group formed by an alkyl group comprising 1 to 12 carbon atoms; an aryl comprising 6 to 20 carbon atoms, said alkyl and aryl groups being optionally substituted.

4. The method according to claim 1, wherein in the methoxyborane according to formula (I) and the organoborane according to formula (II), R¹ and R² taken together with the boron atom to which they are bound, form a heterocycle comprising 5 to 10 members, said heterocycle being optionally substituted.

5. The method according to claim 1, wherein in the organoborane according to formula (II), X is chosen in the group formed by a hydrogen atom; a carboxylate group having the formula —OCOR⁸ wherein R⁸ is chosen from a hydrogen atom, an alkyl group comprising 1 to 12; a halogen atom; an alkyl group comprising 1 to 12; a sulphonate group having the formula —OSO₂R⁷, wherein R⁷ is chosen from a methyl group (CH₃), a trifluoromethyl group (CF₃), a toluene group (p-CH₃C₆H₄) or a benzene group (C₆H₅).

6. The method according to claim 1, wherein the organoborane according to formula (II) is chosen in the group formed by tributylborane, dicyclohexylborane, iododicyclohexylborane, dibutylborane methanesulphonate (n-Bu₂BOSO₃Me), B-iodo-9-borabicyclo[3.3.1]nonane, the dimer of 9-borabicyclo[3.3.1]nonane, B-benzyl-9-borabicyclo[3.3.1]nonane, Me-TBD-BBN⁺I⁻.

7. The method according to claim 1, wherein the base is an organic base chosen from:
nitrogen-containing organic bases chosen in the group formed by triethylamine, trimethylamine, N-diisopropylethylamine (DIPEA), diethylisopropylamine (DIEA), 7-Methyl-1,5,7-triazabicyclo[4.4.0]dec-5-ene (MTBD), 1,8-diazabicyclo[5.4.0]undec-7-ene (DBU), 1,4-diazabicyclo[2.2.2]octane (DABCO), and N-methylpiperidine;
phosphorus-containing organic bases chosen in the group formed by triphenylphosphine, 2,2′-bis(diphenylphosphino)-1,1′-binaphthyl (BINAP), triisopropylphosphine, 1,2-bis(diphenylphosphino)ethane (dppe), tricyclohexylphosphine (PCy₃); aza-phosphines chosen in the group formed by 2,8,9-triisopropyl-2,5,8,9-tetraaza-1-phosphabicyclo[3.3.3]undecane (BV^{Me}) and 2,8,9-triisobutyl-2,5,8,9-tetraaza-1-phosphabicyclo[3.3.3]undecane (BV^{iBu});
carbon-containing bases chosen from the N-heterocyclic carbenes derived from an imidazolium salt, said carbenes being chosen in the group formed by the salts of 1,3-bis(2,6-diisopropylphenyl)-1H-imidazol-3-ium (also referred to as IPr), 1,3-bis(2,6-diisopropylphenyl)-4,5-dihydro-1H-imidazol-3-ium (also referred to as s-IPr), 1,3-bis(2,4,6-trimethylphenyl)-1H-imidazol-3-ium (also referred to as IMes), 1,3-bis(2,4,6-trimethylphenyl)-4,5-dihydro-1H-imidazol-3-ium (also s-IMes), 4,5-dichloro-1,3-bis(2,6-diisopropylphenyl)-1H-imidazol-3-ium (also referred to as Cl₂IPr), 1,3-di-tert-butyl-1H-imidazol-3-ium (also referred to as ItBu), and 1,3-di-tert-butyl-4,5-dihydro-1H-imidazol-3-ium (also referred to as s-ItBu), said salts being in the form of chloride or tetraphenylborate salts.

8. The method according to claim 1, wherein the organic base is a nitrogen-containing organic base chosen in the group formed by triethylamine, trimethylamine, N-diisopropylethylamine (DIPEA), diethylisopropylamine (DIEA), 7-Methyl-1,5,7-triazabicyclo[4.4.0]dec-5-ene (MTBD), 1,8-diazabicyclo[5.4.0]undec-7-ene (DBU), 1,4-diazabicyclo[2.2.2]octane (DABCO), and N-methylpiperidine.

9. The method according to claim 1, wherein the dismutation of formic acid, or of at least one of the derivatives thereof or of a mixture of formic acid and at least one of the derivatives thereof as defined in claim 1, further takes place in the presence of an additive chosen from:
crown ethers in the group formed by 12-crown-4, 15-crown-5, 18-crown-6, dibenzo-18-crown-6, benzo-18-crown-6, benzo-15-crown-5, and dibenzo-15-crown-5;
aza-crowns in the group formed by 1,4,7,10-tetraazacyclododecane (cyclene), 1,4,7,10,13,16-hexaazacyclooctadecane (hexacyclene), and diaza-18-crown-6;
crown thioethers in the group formed by 1,5,9,13-tetrathiacyclohexadecane (16-Ane-S₄), and 1,4,7,10,13,16-hexathiacyclooctadecane (18-Ane-S₆).

10. The method according to claim 1, wherein the quantity of the organoborane according to formula (II) is 0.05 to 1 molar equivalents, inclusive, with respect to formic acid or the derivative(s) thereof, or a mixture of formic acid and at least one of the derivatives thereof.

11. The method according to claim 1, wherein the base quantity is 0.05 to 3 molar equivalents, inclusive, with respect to the formic acid derivative(s), or a mixture of formic acid and at least one of the derivatives thereof.

12. The method according to claim 1, wherein the quantity of additive is 1 to 2 molar equivalents, inclusive, with respect to the formic acid derivative(s), or a mixture of formic acid and at least one of the derivatives thereof.

13. A method for preparing methanol wherein it comprises
(A) a step for preparing a methoxyborane according to formula (I) by dismutation of formic acid, or of at least one of the derivatives thereof having the formula HCO₂M wherein M is chosen in the group formed by Na⁺, K⁺, Li⁺, Cs⁺, NH₄⁺, triethylammonium (HNEt₃⁺), tetraphenylphosphonium (PPh₄⁺), tetramethylammonium (NMe₄⁺), tetraethylammonium (NEt₄⁺), tetrabutylammonium (NBu₄⁺), and tetraphenylammonium (NPh₄⁺), or of a mixture of formic acid and at least one of the derivatives thereof,
in the presence
of an organoborane according to formula (II); and optionally
of an organic or inorganic base,
according to claim 1; and
(B) a step for the hydrolysis or protonolysis of the methoxyborane according to formula (I) obtained following the dismutation step (A) into methanol.

14. A method for producing methanol wherein it comprises
(i) a step for preparing formic acid or the derivatives thereof having the formula HCO₂M wherein M is chosen in the group formed by Na⁺, K⁺, Li⁺, Cs⁺, NH₄⁺, triethylammonium (HNEt₃⁺), tetraphenylphosphonium (PPh₄⁺), tetramethylammonium (NMe₄⁺), tetraethylammonium (NEt₄⁺), tetrabutylammonium (NBu₄⁺), and tetraphenylammonium (NPh₄⁺), by 2e⁻ reduction of CO₂, or by catalytic hydrogenation of CO₂;
(ii) a step for preparing a methoxyborane according to formula (I) by dismutation of formic acid or of at least one of the derivatives thereof, or of a mixture of formic acid and at least one of the derivatives thereof, in presence of an organoborane according to formula (II), and optionally an organic or inorganic base, according to claim 1; and
(iii) a step for the hydrolysis or protonolysis of the methoxyborane according to formula (I) obtained following the dismutation step (ii) into methanol.
